(19) United States
(12) Patent Application Publication
(10) Pub. No.: US 2025/0207036 A1
(43) Pub. Date: Jun. 26, 2025

(54) METHODS TO PRODUCE HYDROCARBONS FROM CARBON DIOXIDE

(71) Applicant: West Virginia University Board of Governors on Behalf of West Virginia University, Morgantown, WV (US)

(72) Inventors: Jianli HU, Morgantown, WV (US); Sonit BALYAN, Vancouver (CA); Brandon ROBINSON, Morgantown, WV (US)

(21) Appl. No.: 19/001,687

(22) Filed: Dec. 26, 2024

Related U.S. Application Data
(60) Provisional application No. 63/614,841, filed on Dec. 26, 2023.

(51) Int. Cl. C10G 3/00; C07C 29/153

(52) U.S. Cl. CPC C10G 3/49; C07C 29/153; C07C 2523/06; C07C 2523/08; C07C 2523/26; C10G 2300/4006; C10G 2300/4012; C10G 2400/02; C10G 2400/04; C10G 2400/08; C10G 2400/20; C10G 2400/22

(57) ABSTRACT

In one aspect, the disclosure relates to a method comprising: flowing a gas mixture over a first catalyst; heating the first catalyst by microwave irradiation to a first target temperature, thereby producing a first gaseous product comprising methanol; flowing the first gaseous product over a molecular sieve catalyst; heating the molecular sieve catalyst to a second target temperature, thereby producing a second product comprising at least one hydrocarbon compound. The disclosure also relates to compositions produced using the disclosed methods. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/614,841, filed on Dec. 26, 2023, which is incorporated herein by reference in its entirety.

### BACKGROUND

CO₂ is a highly stable molecule that requires the input of energy for its transformation and utilization. The direct conversion of CO₂ into valuable chemicals has emerged as a strategic approach to address the rise in atmospheric CO₂ level and utilize it as a carbon source. The process of CO₂ hydrogenation into olefins is one approach to produce carbon-based fuels and feedstock chemicals. However, the selective hydrogenation of CO₂ to produce compounds containing two or more carbon atoms has significant challenges, stemming at least in part from the inherent inertness of CO₂ and the substantial energy barrier associated with selective carbon-carbon coupling processes. One potential approach to produce light olefins from CO₂ is a two-stage process using a catalyst that includes CO₂ conversion to methanol followed by methanol conversion to olefins. The conversion of CO₂ to methanol is hindered by the unavoidable production of water vapor, which can hamper the reaction and results in substantial degradation of the catalyst. Despite advances in catalyst research, there is still a need for methods and systems for efficient and cost-effective productions of olefins and fuels from CO₂. These needs and other needs are satisfied by the present disclosure.

### SUMMARY

In accordance with the purpose(s) of the present disclosure, as embodied and broadly described herein, the disclosure, in one aspect, relates methods for production of hydrocarbon compounds from carbon dioxide. A method, comprising: flowing a gas mixture over a first catalyst; heating the first catalyst by microwave irradiation to a first target temperature, thereby producing a first gaseous product comprising methanol; flowing the first gaseous product over a molecular sieve catalyst; heating the molecular sieve catalyst to a second target temperature, thereby producing a second product comprising at least one hydrocarbon compound. The gas mixture can comprise CO₂ and H₂. The first catalyst can comprise a first metallic nanoparticle and a second metallic nanoparticle on a support, where the first metallic nanoparticle is selected from Cu, Cr, Co, Pd, and a combination thereof; the second metallic nanoparticle is selected from ZnO, Cr₂O₃, In₂O₃, and a combination thereof; and the support comprises Al₂O₃, MgO, SiO₂—Al₂O₃, or Cr₂O₃. The disclosure also relates to hydrocarbon compounds (e.g., olefins, alcohols, and fuels) made by the disclosed systems and methods.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic depicting representative reactor configurations for a modular catalyst system.

FIGS. 2A-2B show representative data pertaining to the effect of pressure on conversion (FIG. 2A) and product selectivity (FIG. 2B) with process conditions of: temperature—160° C.; catalyst weight—1.5 gm; microwave power—0.4 KW; H₂:CO₂ ratio—3:1; flow rate—12.5 sccm.

FIGS. 3A-3B show representative data pertaining to the effect of temperature on methanol production and H₂ and CO₂ conversion at 60-minute TOS (FIG. 3A), and 120-minute TOS (FIG. 3B) with process conditions of: pressure—80 psig; catalyst weight—1.5 gm; microwave power—0.4 KW; H₂:CO₂ ratio—3:1; flow rate—12.5 sccm.

FIGS. 4A-4C show representative data pertaining to the effect of temperature on CO₂ conversion (FIG. 4A), H₂ conversion (FIG. 4B), and methanol production (FIG. 4C) with process conditions of: pressure—80 psig; catalyst weight—1.5 gm; H₂:CO₂ ratio—3:1; flow rate—12.5 sccm.

FIGS. 5A-5B show catalytic performance of a representative Cu/ZnO/Al₂O₃ catalyst for CO₂ hydrogenation by CO₂ and H₂ conversion (FIG. 5A) and methanol production (FIG. 5B) with process conditions of: pressure, conventional heating—362 psig; temperature, conventional heating—240° C.; pressure, microwave heating—80 psig; temperature, microwave heating—200° C.; catalyst weight—1.5 gm; microwave power—0.4 KW; H₂:CO₂ ratio—3:1; flow rate—12.5 sccm.

FIG. 6A shows representative catalytic performance of Cu/ZnO/Al₂O₃ and SAPO-34 catalyst for CO₂ hydrogenation to olefins after 60 minutes with process conditions of: pressure-80 psig; catalyst weight—1.5 gm Cu/ZnO/Al₂O₃, 0.3 gm SAPO-34; microwave power—0.4 KW; H₂:CO₂ ratio—3:1; flow rate—12.5 sccm.

FIG. 6B shows catalytic performance of Cu/ZnO/Al₂O₃+SAPO-34 catalyst for CO₂ hydrogenation to olefins with process conditions of: pressure—80 psig; temperature—200° C.; catalyst weight—1.5 gm Cu/ZnO/Al₂O₃, 0.3 gm SAPO-34; microwave power—0.4 KW; H₂:CO₂ ratio—3:1; flow rate—12.5 sccm.

FIG. 7 shows representative X-ray diffraction spectra of a calcined and a spent Cu/ZnO/Al₂O₃ catalyst.

FIG. 8 shows a representative temperature programmed reduction profile of a calcined Cu/ZnO/Al₂O₃ catalyst.

FIG. 9 shows representative data pertaining to the effect of an MTO reaction temperature on ethylene production.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an olefin,” “a fuel,” or “a nanoparticle,” including, but not limited to, two or more such olefins, fuels, or nanoparticles, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a temperature refers to an amount that is sufficient to achieve the desired improvement in the property modulated by the formulation component, e.g. achieving the desired level of modulus. The specific level in terms of wt % in a composition required as an effective amount will depend upon a variety of factors including the amount and type of olefin that is desired, amount and type of fuel that is desired, and economic considerations.

As used herein, the terms “optional” or “optionally” means that the subsequently described event or circumstance can or cannot occur, and that the description includes instances where said event or circumstance occurs and instances where it does not.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkene” or “olefin” as used herein refers to a hydrocarbon compound of from 2 to 24 carbon atoms with a structural formula containing at least one carbon-carbon double bond. Asymmetric structures such as (A¹A²) C═C (A³A⁴) are intended to include both the E and Zisomers. This can be presumed in structural formulae herein wherein an asymmetric alkene is present, or it can be explicitly indicated by the bond symbol C═C. An alkene compound can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

As used herein, the term “light olefin” refers to an olefin of 2 to 4 carbon atoms (i.e., a C2-C4 alkene).

Unless otherwise specified, temperatures referred to herein are based on atmospheric pressure (i.e. one atmosphere).

### B. ABBREVIATIONS

MTO methanol-to-olefin
TOS time-on-stream
XRD X-ray diffraction

### C. METHODS FOR CO₂ HYDROGENATION

The direct conversion of CO₂ into valuable chemicals, rather than recognizing it solely as a greenhouse gas, has emerged as a strategic approach to address the rise in atmospheric CO₂ level and utilize it as a carbon source. (Refs. 1-3) As a substitute to conventional fossil-based compounds, the utilization of CO₂ as a raw material for the synthesis of chemicals has gained traction. (Refs. 4-7) Catalytic hydrogenation is one path for directly transforming CO₂ into valuable chemicals and transportation fuels. (Refs. 8-13) Conventional methods of making olefins from carbon dioxide hydrogenation involve undergoing three distinct reactions as follows: (i) reverse water gas shift reaction CO₂+H₂=CO+H₂O; (ii) methanol synthesis: CO+2H₂═CH₃OH; and (iii) methanol to olefin production. These three reactions are each operated under different temperatures. Moreover, the conventional methods are typically carried out at high pressures (e.g., about 20 bar or 290 psi).

Disclosed herein are methods comprising microwave selective heating to convert CO₂ to methanol in a low temperature zone, followed by conversion of methanol to hydrocarbon compounds over a molecular sieve catalyst in a thermally heated zone. The entire process can be carried out in a single tubular reactor. The methods of the present disclosure can be scaled for activating stable molecule CO₂ and converting CO₂ to value added chemicals such as sustainable aviation fuel. In another aspect, the methods and processes of the present disclosure can provide a carbon negative for certain industrial processes. Advantages of the methods and processes of the present disclosure include: (a) scalability for carbon capture and utilization (many carbon capture technologies do not have a solution for CO₂ storage) and (b) operability under low pressures and/or low temperatures in a single reactor. In a further aspect, the microwave energy used in the present disclosure can be coupled to a renewable energy source. In one aspect, the microwave irradiation of the presently disclosed methods and processes can directly interact with the disclosed catalysts to deliver energy onto the catalyst, rather than being used as external heating sources. The benefits of direct energy deposition of microwave include enhanced reaction rate, product selectivity, and low temperature operation.

More specifically, disclosed herein is a method comprising: flowing a gas mixture over a first catalyst; heating the first catalyst by microwave irradiation to a first target temperature, thereby producing a first gaseous product comprising methanol; flowing the first gaseous product over a molecular sieve catalyst; heating the molecular sieve catalyst to a second target temperature, thereby producing a second product comprising at least one hydrocarbon compound. The molecular sieve catalyst can be heated using microwave irradiation, microwave plasma, or conventional thermal heating methods (e.g., using a furnace).

In one aspect, the first catalyst can comprise a first metallic nanoparticle and a second metallic nanoparticle on a support. In one aspect, the gas mixture can comprise CO₂ and H₂. In a further aspect, the gas mixture can comprise a carrier gas. Examples of carrier gases include He, N₂, Ar, combinations thereof, and the like. The gas mixture comprises CO₂ and H₂ in a volume ratio of about 1:10 to about 1:1. The gas mixture can comprise CO₂ and H₂, for example CO₂ and H₂ in a volume ratio of about 1:10 to about 4:5, about 1:10 to about 3:5, 1:10 to about 2:5, or about 5:10 to about 2:5. The first catalyst can comprise a first metallic nanoparticle and a second metallic nanoparticle disposed on a support. The first metallic nanoparticle can be selected from Cu, Cr, Co, Pd, and a combination thereof. In one aspect, second metallic nanoparticle can be selected from ZnO, Cr₂O₃, In₂O₃, and a combination thereof. In another aspect, the support can comprise Al₂O₃, MgO, SiO₂—Al₂O₃, or Cr₂O₃.

The hydrocarbon compounds produced using the methods disclosed herein can include olefins (e.g., light olefins), C5-C25 hydrocarbons, C1-C4 alcohols, and higher alcohols (i.e., alcohols of C5 length or greater). The hydrocarbon compounds produced can include one or more hydrocarbon fuels, in one aspect liquid hydrocarbon fuels, including gasoline (e.g., C4 to C12 hydrocarbons), jet fuel (e.g., C5 to C16 hydrocarbons), and diesel (e.g., C8 to C21 hydrocarbons).

Compared to traditional methods of CO₂ hydrogenation, the method disclosed herein can be carried out at reduced pressure and temperatures. In one aspect, the method can be performed at a pressure of at least about 1 bar. In a further aspect, the method can be performed at a pressure of about 1 bar to about 100 bar, about 1 bar to about 75 bar, about 1 bar to about 50 bar, or about 1 bar to about 25 bar. The first target temperature can be greater than about 160° C., greater than about 170° C., greater than about 180° C., greater than about 190° C., or greater than about 200° C. In another aspect, the first target temperature can be from about 160° C. to about 500° C., about 160° C. to about 450° C., about 160° C. to about 400° C., about 160° C. to about 350° C., about 160° C. to about 300° C., about 180° C. to about 500° C., about 180° C. to about 450° C., or about 180° C. to about 400° C. In another aspect, the second target temperature can be from about 380° C. to about 500° C., about 380° C. to about 450° C., or about 400° C. to about 500° C.

In one aspect, the method can include the use of a molecular sieve catalyst. Molecular sieves are porous solids comprising pores of different sizes. Porse sizes in molecular sieves can range from diameters of less than 5.0 Å (small pore molecular sieve), 5.0 to 10.0 Å (medium pore molecular sieve), or greater than 10.0 Å (large pore molecular sieve). Molecular sieves can be naturally formed (e.g., mineral molecular sieves) or synthetically formed. Molecular sieves can include zeolitic-type molecular sieves. A zeolite is a crystalline aluminosilicate material that can be naturally or synthetically formed. Examples of zeolitic molecular sieves include silicoaluminophosphate (SAPO) molecular sieves or zeolite Socony Mobil (ZSM) molecular sieves (e.g., ZSM-4 and ZSM-5). Examples of SAPO molecular sieves include SAPO-18, SAPO-34, SAPO-35, SAPO-44, and SAPO-56.

### D. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
Ref 1 M. D. Porosoff, B. Yan and J. G. Chen, Energy Environ. Sci., 2016, 9, 62-73.
Ref. 2 U. Rodemerck, M. Holeňa, E. Wagner, Q. Smejkal, A. Barkschat and M. Baerns, ChemCatChem, 2013, 5, 1948-1955.
Ref 3 W. Wang, S. Wang, X. Ma and J. Gong, Chem. Soc. Rev., 2011, 40, 3703-3727.
Ref 4 S. Li, Y. Xu, Y. Chen, W. Li, L. Lin, M. Li, Y. Deng, X. Wang, B. Ge, C. Yang, S. Yao, J. Xie, Y. Li, X. Liu and D. Ma, Angew. Chemie—Int. Ed., 2017, 56, 10761-10765.
Ref 5 X. Wang, G. Yang, J. Zhang, S. Chen, Y. Wu, Q. Zhang, J. Wang, Y. Han and Y. Tan, Chem. Commun., 2016, 52, 7352-7355.
Ref 6 M. Aresta, A. Dibenedetto and E. Quaranta, J. Catal., 2016, 343, 2-45.
Ref 7 X. Wang, Y. Wang, B. Robinson, Q. Wang and J. Hu, J. Catal., 2022, 413, 138-149.
Ref 8 M. D. Porosoff, X. Yang, J. A. Boscoboinik and J. G. Chen, Angew. Chemie—Int. Ed., 2014, 53, 6705-6709.
Ref 9 X. Yang, S. Kattel, S. D. Senanayake, J. A. Boscoboinik, X. Nie, J. Graciani, J. A. Rodriguez, P. Liu, D. J. Stacchiola and J. G. Chen, J. Am. Chem. Soc., 2015, 137, 10104-10107.
Ref 10 J. Graciani, K. Mudiyanselage, F. Xu, A. E. Baber, D. G. Evans, S. D. Senanayake, D. J. Stacchiola, P. Liu, J. Hrbek, J. F. Sanz and J. A. Rodriguez, Science, 2014, 345, 546-550.
Ref 11 G. Centi and S. Perathoner, Catal. Today, 2009, 148, 191-205.
Ref 12 J. Wei, Q. Ge, R. Yao, Z. Wen, C. Fang, L. Guo, H. Xu and J. Sun, Nat. Commun., 2017, 8, 1-8.
Ref 13 F. Wang, S. He, H. Chen, B. Wang, L. Zheng, M. Wei, D. G. Evans and X. Duan, J. Am. Chem. Soc., 2016, 138, 6298-6305.
Ref 14 J. Li, Y. Qi, Z. Liu, G. Liu and D. Zhang, Catal. Letters, 2008, 121, 303-310.
Ref 15 P. Gao, S. Dang, S. Li, X. Bu, Z. Liu, M. Qiu, C. Yang, H. Wang, L. Zhong, Y. Han, Q. Liu, W. Wei and Y. Sun, ACS Catal., 2018, 8, 571-578.
Ref 16 B. Paweelec, R. Guil-Lopes, N. Mota, J. L. G. Fierro and R. M. N. Yerga, Materials (Basel)., 2021, 14, 6952.
Ref 17 O. A. Ojelade and S. F. Zaman, J. CO2 Util., 2021, 47, 101506.
Ref 18 Y. Liu, B. Chen, R. Liu, W. Liu, X. Gao, Y. Tan, Z. Zhang and W. Tu, Fuel, 2022, 324, 124649.
Ref 19 X. Wang, D. Wu, J. Zhang, X. Gao, Q. Ma, S. Fan and T. S. Zhao, Appl. Catal. A Gen., 2019, 573, 32-40.
Ref 20 S. Wang, T. Wu, J. Lin, Y. Ji, S. Yan, Y. Pei, S. Xie, B. Zong and M. Qiao, ACS Catal., 2020, 10, 6389-6401.
Ref 21 A. Ramirez, A. Dutta Chowdhury, M. Caglayan, A. Rodriguez-Gomez, N.

Wehbe, E. Abou-Hamad, L. Gevers, S. Ould-Chikh and J. Gascon, Catal. Sci. Technol., 2020, 10, 1507-1517.
Ref 22 T. Numpilai, N. Chanlek, Y. Poo-Arporn, S. Wannapaiboon, C. K. Cheng, N. Siri-Nguan, T. Sornchamni, P. Kongkachuichay, M. Chareonpanich, G. Rupprechter, J. Limtrakul and T. Witoon, Appl. Surf. Sci., 2019, 483, 581-592.
Ref 23 T. Numpilai, N. Chanlek, Y. Poo-Arporn, C. K. Cheng, N. Siri-Nguan, T. Sornchamni, M. Chareonpanich, P. Kongkachuichay, N. Yigit, G. Rupprechter, J. Limtrakul and T. Witoon, ChemCatChem, 2020, 12, 3306-3320.
Ref 24 A. Dokania, A. Dutta Chowdhury, A. Ramirez, S. Telalovic, E. Abou-Hamad, L. Gevers, J. Ruiz-Martinez and J. Gascon, J. Catal., 2020, 381, 347-354.
Ref 25 J. Ding, L. Huang, W. Gong, M. Fan, Q. Zhong, A. G. Russell, H. Gu, H. Zhang, Y. Zhang and R. ping Ye, J. Catal., 2019, 377, 224-232.
Ref 26 A. Ramirez, L. Gevers, A. Bavykina, S. Ould-Chikh and J. Gascon, ACS Catal., 2018, 8, 9174-9182.
Ref 27 B. Liang, J. Ma, X. Su, C. Yang, H. Duan, H. Zhou, S. Deng, L. Li and Y. Huang, Ind. Eng. Chem. Res., 2019, 58, 9030-9037.
Ref 28 X. Zhai, J. Shamoto, H. Xie, Y. Tan, Y. Han and N. Tsubaki, Fuel, 2008, 87, 430-434.
Ref 29 P. Tian, Y. Wei, M. Ye and Z. Liu, ACS Catal., 2015, 5, 1922-1938.
Ref 30 H. Fu, W. Song and J. F. Haw, Catal. Letters, 2001, 76, 89-94.
Ref 31 E. S. P. B. V, G. C. Chinchen, P. J. Denny, J. R. Jennings, K. C. Waugh and P. Group, 1988, 36, 1-65.
Ref 32 F. Zhang, X. Xu, Z. Qiu, B. Feng, Y. Liu, A. Xing and M. Fan, Green Energy Environ., 2022, 7, 772-781.
Ref 33 B. Hu, Y. Yin, G. Liu, S. Chen, X. Hong and S. C. E. Tsang, J. Catal., 2018, 359, 17-26.
Ref 34 B. S. Clausen, G. Steffensen, B. Fabius, J. Villadsen, R. Feidenhans'l and H. Topsøe, J. Catal., 1991, 132, 524-535.
Ref 35 J. Yoshihara and C. T. Campbell, J. Catal., 1996, 161, 776-782.

### E. ASPECTS

The following listing of exemplary aspects supports and is supported by the disclosure provided herein.

Aspect 1. A method, comprising: flowing a gas mixture over a first catalyst; heating the first catalyst by microwave irradiation to a first target temperature, thereby producing a first gaseous product comprising methanol; flowing the first gaseous product over a molecular sieve catalyst; heating the molecular sieve catalyst to a second target temperature, thereby producing a second product comprising at least one hydrocarbon compound; wherein the gas mixture comprises CO₂ and H₂; wherein the first catalyst comprises a first metallic nanoparticle and a second metallic nanoparticle on a support; wherein the first metallic nanoparticle is selected from Cu, Cr, Co, Pd, and a combination thereof; wherein the second metallic nanoparticle is selected from ZnO, Cr₂O₃, In₂O₃, and a combination thereof; and wherein the support comprises Al₂O₃, MgO, SiO₂—Al₂O₃, or Cr₂O₃.

Aspect 2. The method of aspect 1, wherein the method is carried out a pressure of about 1 bar to about 100 bar.

Aspect 3. The method of aspect 1, wherein the method is carried out a pressure of about 1 bar to about 75 bar.

Aspect 4. The method of aspect 1, wherein the method is carried out a pressure of about 1 bar to about 50 bar.

Aspect 5. The method of aspect 1, wherein the method is carried out a pressure of about 1 bar to about 25 bar.

Aspect 6. The method of any one of aspects 1-5, wherein the molecular sieve catalyst is a zeolitic molecular sieve catalyst.

Aspect 7. The method of aspect 6, wherein the zeolitic molecular sieve catalyst comprises SAPO-18, SAPO-34, SAPO-35, SAPO-44, SAPO-56, ZSM-5, or a combination thereof.

Aspect 8. The method of any one of aspects 1-7, wherein the first metallic nanoparticle comprises Cu.

Aspect 9. The method of any one of aspects 1-8, wherein the second metallic nanoparticle is selected from ZnO, Cr₂O₃, and a combination thereof.

Aspect 10. The method of any one of aspects 1-9, wherein the support comprises Al₂O₃ or SiO₂—Al₂O₃.

Aspect 11. The method of any one of aspects 1-10, wherein the first target temperature is greater than about 160° C.

Aspect 12. The method of any one of aspects 1-10, wherein the first target temperature is from about 160° C. to about 500° C.

Aspect 13. The method of any one of aspects 1-10, wherein the first target temperature is from about 160° C. to about 400° C.

Aspect 14. The method of any one of aspects 1-10, wherein the first target temperature is from about 180° C. to about 400° C.

Aspect 15. The method of any one of aspects 1-14, wherein the second target temperature is from about 380° C. to about 500° C.

Aspect 16. The method of any one of aspects 1-14, wherein the second target temperature is from about 400° C. to about 500° C.

Aspect 17. The method of any one of aspects 1-16, wherein the gas mixture comprises CO₂ and H₂ in a volume ratio of about 1:10 to about 1:1.

Aspect 18. The method of any one of aspects 1-16, wherein the gas mixture comprises CO₂ and H₂ in a volume ratio of about 1:10 to about 4:5.

Aspect 19. The method of any one of aspects 1-16, wherein the gas mixture comprises CO₂ and H₂ in a volume ratio of about 1:10 to about 3:5.

Aspect 20. The method of any one of aspects 1-16, wherein the gas mixture comprises CO₂ and H₂ in a volume ratio of about 1:10 to about 2:5.

Aspect 21. The method of any one of aspects 1-16, wherein the gas mixture comprises CO₂ and H₂ in a volume ratio of about 5:10 to about 2:5.

Aspect 22. The method of any one of aspects 1-21, wherein the gas mixture further comprises a carrier gas selected from He, N₂, Ar, and a combination thereof.

Aspect 23. The method of any one of aspects 1-22, wherein the at least one hydrocarbon compound comprises an olefin.

Aspect 24. The method of any one of aspects 1-23, wherein the at least one hydrocarbon compound comprises a light olefin.

Aspect 25. The method of any one of aspects 1-24, wherein the at least one hydrocarbon compound comprises a C5-C25 hydrocarbon.

Aspect 26. The method of any one of aspects 1-25, wherein the at least one hydrocarbon compound comprises a C₁-C₄ alcohol.

Aspect 27. The method of any one of aspects 1-26, wherein the at least one hydrocarbon compound comprises a higher alcohol.

Aspect 28. The method of any one of aspects 1-27, wherein the at least one hydrocarbon compound comprises a hydrocarbon fuel.

Aspect 29. The method of any one of aspects 1-27, wherein the at least one hydrocarbon compound comprises a hydrocarbon fuel selected from gasoline, jet fuel, diesel, and a combination thereof.

Aspect 30. A composition, comprising an olefin produced using the method of any one of aspects 1-29.

Aspect 31. A composition, comprising a hydrocarbon fuel produced using the method of any one of the aspects of 1-29.

Aspect 32. The composition of aspect 31, wherein the hydrocarbon fuel is selected from gasoline, jet fuel, and diesel.

Aspect 33. A catalyst, comprising: a support; a first metallic nanoparticle dispersed on the support; and a second metallic nanoparticle dispersed on the support; wherein the first metallic nanoparticle is selected from Cu, Cr, Co, Pd, and a combination thereof; wherein the second metallic nanoparticle is selected from ZnO, Cr₂O₃, In₂O₃, and a combination thereof; and wherein the support comprises Al₂O₃, MgO, SiO₂—Al₂O₃, or Cr₂O₃

Aspect 34. The catalyst of aspect 33, wherein the first metallic nanoparticle comprises Cu.

Aspect 35. The catalyst of aspect 33 or aspect 34, wherein the second metallic nanoparticle is selected from ZnO, Cr₂O₃, and a combination thereof.

Aspect 36. The catalyst of any one of aspects 33-35, wherein the support comprises Al₂O₃ or SiO₂—Al₂O₃.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### F. EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. CO₂ Hydrogenation to Olefins

CO₂ is a highly stable molecule that requires the input of energy for its transformation and utilization. Utilization of renewable sources such as solar and wind to derive hydrogen for catalytic CO₂ hydrogenation is an attractive method for facilitating this conversion process. A wide array of catalysts, encompassing different active metals, has the capability to produce a diverse range of products by the hydrogenation of CO₂. These products include methane, formic acid, formates, hydrocarbons (particularly olefins), alcohols, and fuel oils.

One potential approach to produce light olefins from CO₂ is a two-stage process. Initially, CO₂ is converted into methanol (CH₃OH), which is then further processed to generate olefins via dominant aromatic-based Hydrocarbon pool mechanism proposed by Li and coworkers (Ref. 14) over SAPO-34. The conversion of CO₂ to methanol is hindered by the unavoidable production of water vapor, which significantly hampers the reaction and results in substantial degradation of the catalyst. SAPO-34 zeolite is a favoured catalyst in the conversion of methanol to olefins process due to its distinctive topology, but it is susceptible to quick deactivation caused by the deposition of coke.

Prior studies investigating catalyst deactivation in the methanol synthesis process from CO₂ capture have revealed several aspects that play roles in the conversion processes. (Refs. 15-17) Iron oxide catalysts (Ref. 18) with varying concentrations of sodium (Na) promoters were synthesized and afterwards assessed. The findings indicate that the incorporation of a Na promoter yields a favourable impact on the production of olefins. The ratio of olefins to paraffins, namely in the range of C2-C7, exhibits a significant increase from 0.70 to 5.67 when the concentration of Na promoter is increased from 0% to 0.5%. Fisher-Tropsch catalysts (Ref. 19) having functions of reverse water gas shift reaction enable olefin production via a MeOH or CO intermediate over tandem catalysts. Previous studies have used various catalysts (Refs. 20-26) for CO₂ hydrogenation to selective light olefins. Previous studies (Refs. 27 and 28) reported that several factors affecting CuZnAl catalyst performances, including catalyst poisoning resulting from impurities in the feed gas, sintering of Cu particles at elevated reaction temperatures, and the deposition of carbon on the catalyst surface. These factors have been recognized as contributors to the deactivation phenomenon. The resolution of these concerns could aid in enhancing the industrial feasibility of CO₂ conversion methodologies.

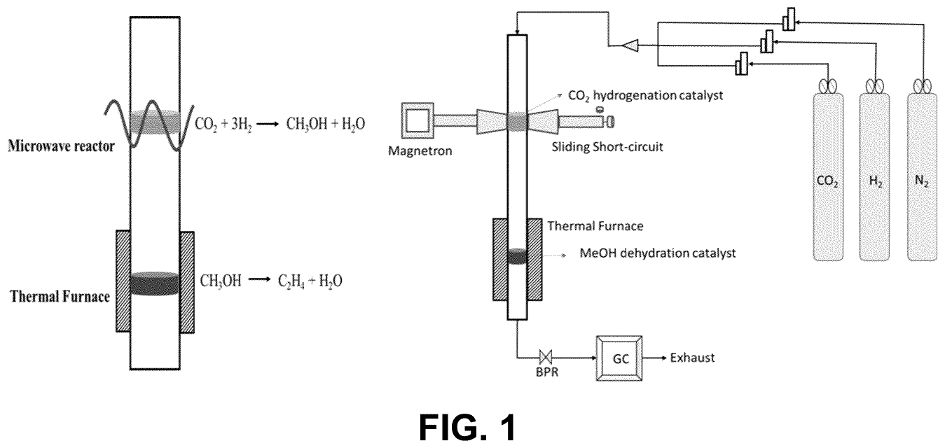

Herein is introduced a methodology for the direct synthesis of light olefins from CO₂ utilizing bimetallic catalyst supported on alumina Cu/ZnO/Al₂O₃, that is responsible for CO₂ activation and other zeolite catalyst SAPO-34 for selective C—C coupling, respectively. The innovative, effective process microwave and thermal heating together is used for the first time for direct conversion of CO₂ into light olefins operated at low pressure and temperature in comparison to other commercialized processes. The reactor scheme of modular catalysis system is shown in FIG. 1. The procedure demonstrated significant selectivity and sensitivity to alcohols and C2-C4 hydrocarbons, meanwhile effectively restricting the methane content. While using this advanced process, negligible coke deposition was observed on Cu/ZnO/Al₂O₃, confirmed by XRD. This offers a pathway for the environmentally friendly synthesis of valuable hydrocarbons, addressing the issue of greenhouse gas emissions. The criteria for catalyst synthesis and experimental details are described below.

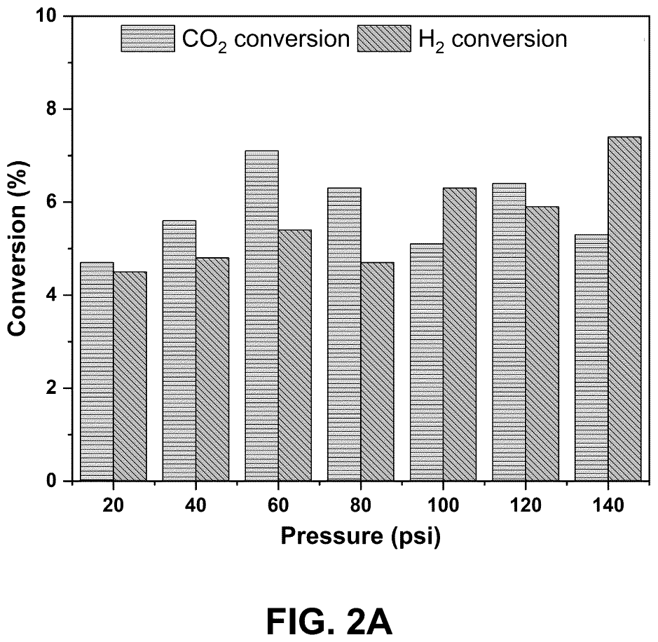

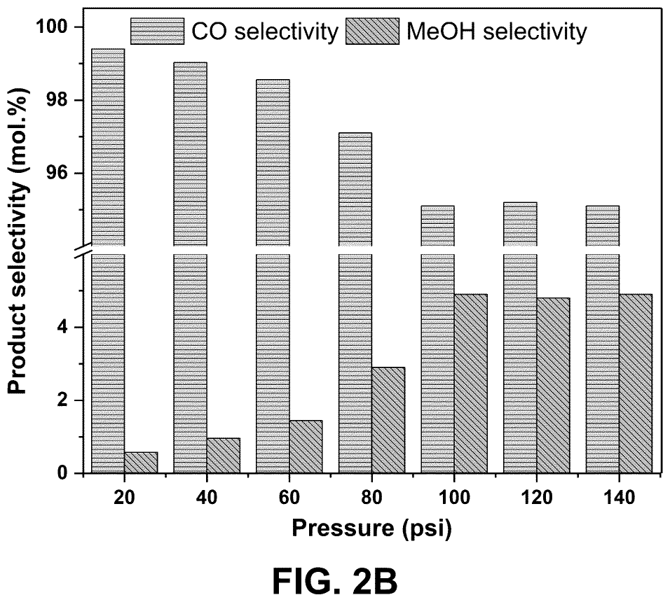

The effect of pressure for methanol synthesis is carried out in a microwave heated packed bed reactor system over CO₂ hydrogenation catalyst under a constant temperature of 160° C. FIGS. 2A-2B show the pressure effect in the product selectivity at 40-minute TOS. An increase in methanol selectivity is observed with an increase in pressure. When the pressure is increased to 140 psig, methanol selectivity reaches 5.1 mol. %, with 94.9 mol. % carbon monoxide selectivity. Additionally, CO₂ and H₂ conversion reaches to 5.3% and 7.4%, respectively. Whereas the methanol selectivity (with 0.6 mol. %) is minimum when the reaction is carried out at 20 psig pressure. Almost no change in methanol selectivity (varying between 4.9 mol. % and 5.1 mo. %) is observed when the reaction pressure is varied from 100 psig to 140 psig, respectively. In this study, the reaction pressure of 80 psig was chosen to carry out further studies to investigate the effect of temperature on the CO₂ hydrogenation to methanol synthesis.

The higher pressures above 80 psig were not chosen because of the safety concerns in the current microwave reactor using a quartz tube. CO₂ hydrogenation to olefins was carried in a modular reactor. For the MTO reaction, a higher temperature in the range of 400° C. to 450° C. is required. (Refs. 29 and 30) To investigate the impact of temperature and other factors, the constant pressure of 80 psig was maintained for further studies.

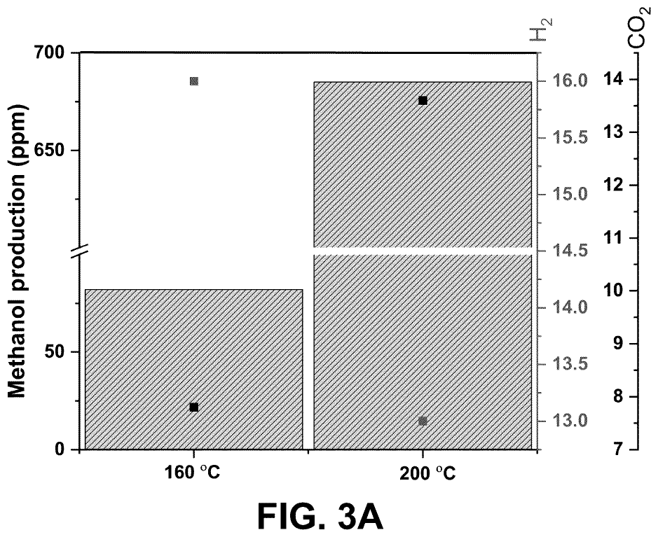

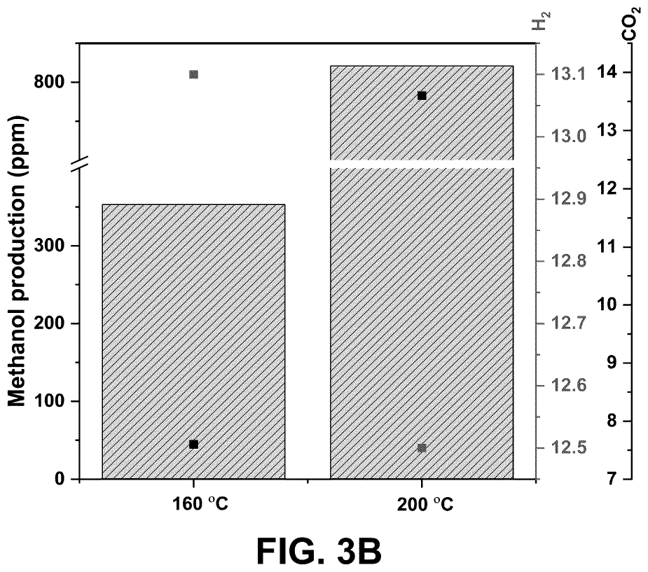

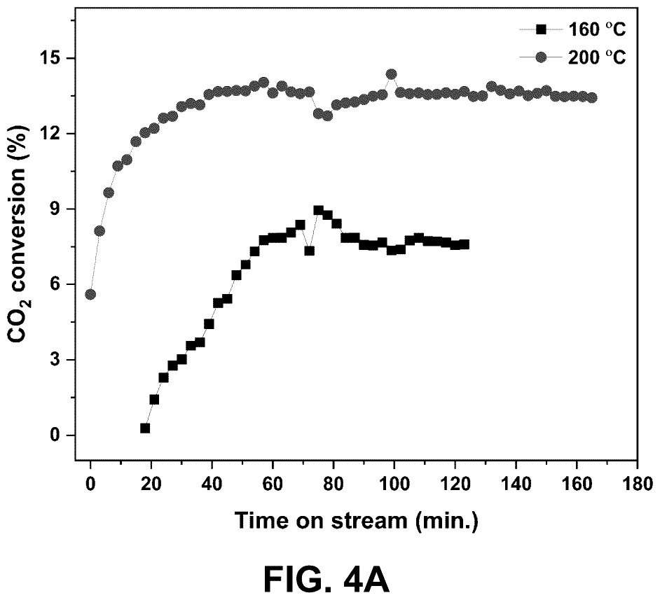

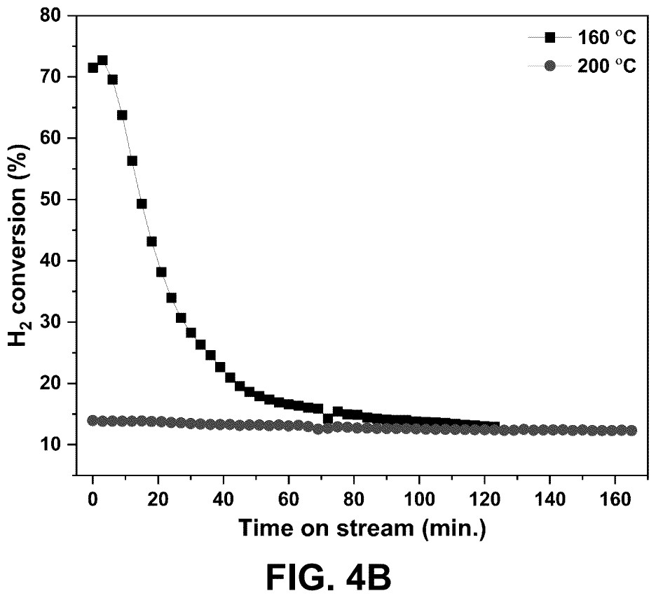

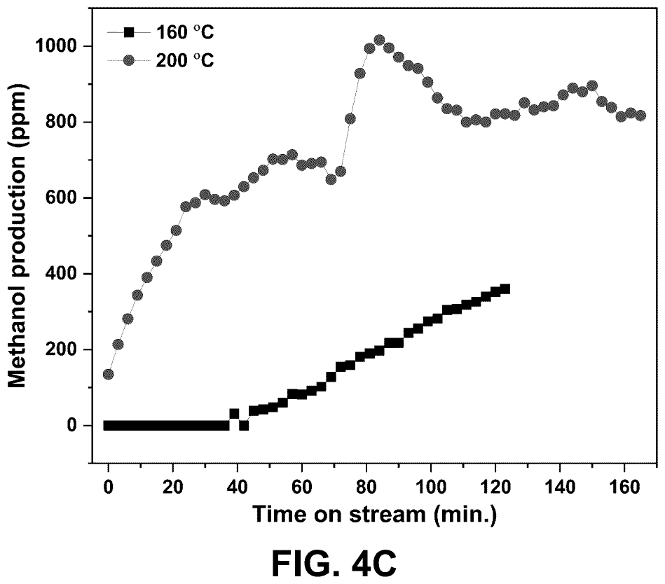

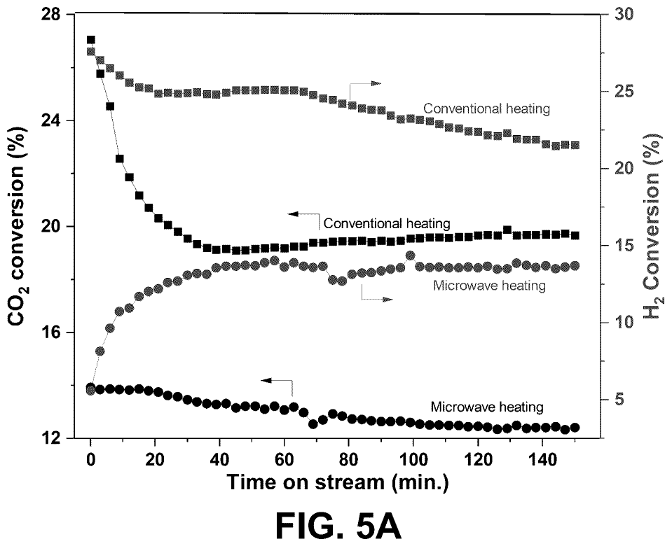

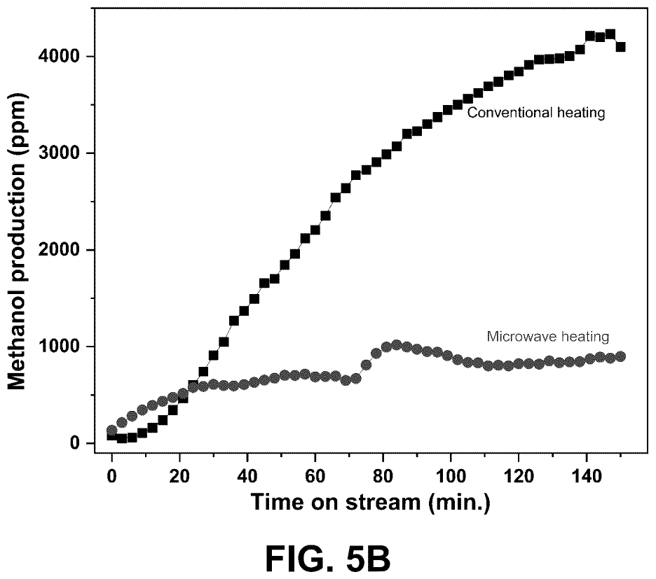

To optimize the reaction temperature for methanol synthesis, experiments of temperature effect were carried out at 80 psig pressure over the Cu/ZnO/Al₂O₃ catalyst bed under microwave heating mode. Two different temperatures (160° C. and 200° C.) were selected for testing (see Table 1). As shown in FIGS. 3A-3B, at 160° C. methanol concentration of 353 ppm is observed at 120-minute TOS with a 7.6% and 13.1% CO₂ and H₂ conversion, respectively. When the reaction temperature is increased to 200° C. the production of methanol was increased from 82 ppm to 685 ppm at 60-minute TOS, and 353 ppm to 821 ppm at 120-minute TOS respectively. The maximum production of methanol (1015 ppm) is observed at 90-minute TOS for the CO₂ hydrogenation catalyst at 200° C. temperature as shown in FIG. 4C. H₂ and CO₂ conversion for the CO₂ hydrogenation catalyst are shown in FIG. 4A and FIG. 4B, respectively. From the study, 200° C. is chosen as the reaction temperature for CO₂ hydrogenation reaction in the further reactions. To compare the effect of microwave heating on the CO₂ hydrogenation to methanol, the hydrogenation reaction is also carried out at 200° C. and 80 psig under conventional furnace heating over the Cu/ZnO/Al₂O₃ catalyst. However, no methanol is observed in the product stream. Here, a comparison between conventional furnace heating (240° C. and 362 psig) and microwave heating (200° C. and 80 psig) CO₂ hydrogenation to methanol is shown in FIGS. 5A-5B.

TABLE 1
Effect of reaction temperature on Methanol production.
60-minute TOS

| | |
|---|---|
| Conversion (%) | Production (ppm) |

| Temperature (° C.) | CO₂ | H₂ | MeOH | CO |
|---|---|---|---|---|
| 160 | 7.8 | 16 | 82 | 1282 |
| 200 | 13.6 | 13 | 685 | 15491 |

120-minute TOS

| | CO₂ | H₂ | MeOH | CO |
|---|---|---|---|---|
| 160 | 7.6 | 13.1 | 353 | 15845 |
| 200 | 13.6 | 12.5 | 821 | 19309 |

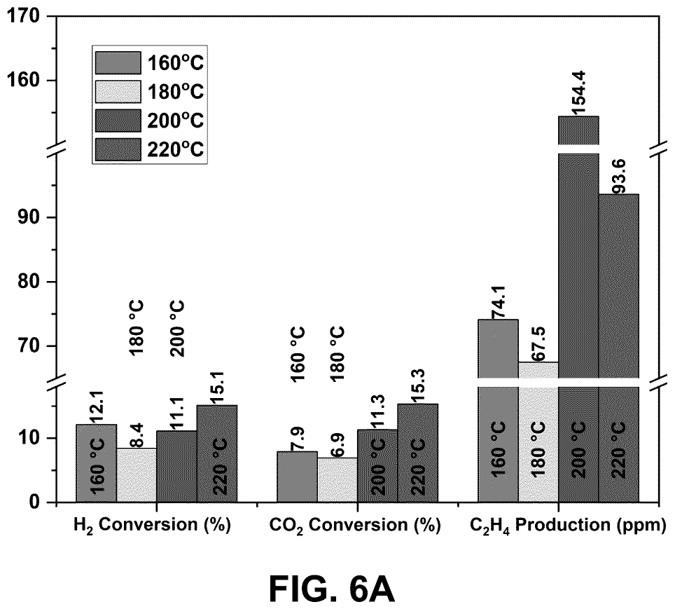

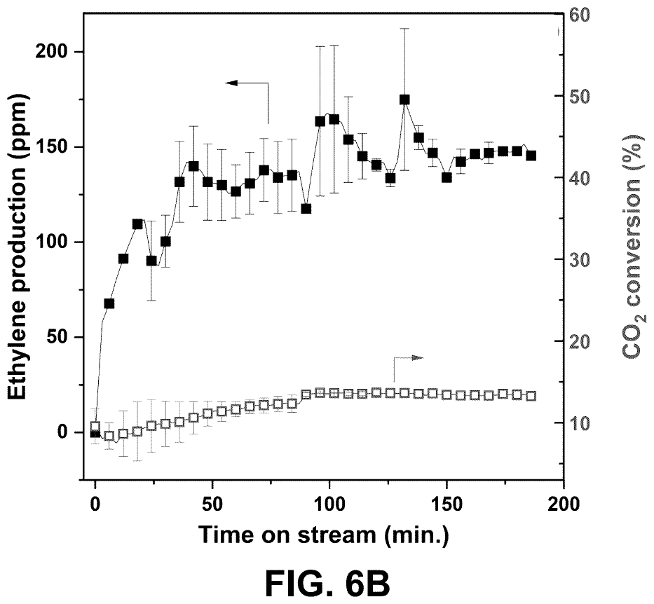

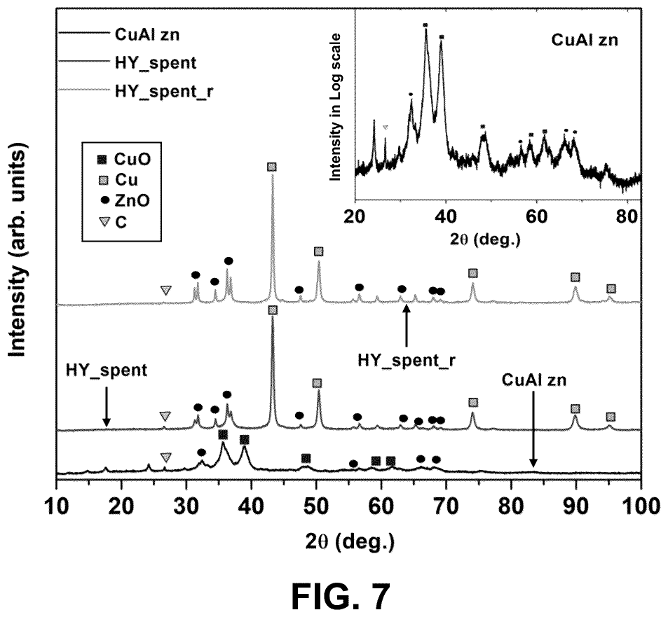

Upon optimizing the process and reactor parameters for CO₂ hydrogenation to methanol, catalytic performance for CO₂ hydrogenation to olefins was carried out in the modular reactor. Another set of experiments to study the effect of microwave reactor temperature was performed at constant 80 psig pressure. SAPO-34 zeolite was heated using a conventional thermal furnace at 425° C. and is used as an MTO catalyst. FIG. 6A shows the catalytic performance of Cu/ZnO/Al₂O₃ and SAPO-34 catalyst. The highest CO₂ and H₂ conversion of about ˜16% and ˜16% respectively are observed when the Cu/ZnO/Al₂O₃ is heated at 220° C. under microwave irradiation and the ethylene production is 93 ppm at 60 min TOS, whereas for the catalyst heated at 200° C., the ethylene production is 160 ppm at 60 minutes TOS. The decrease in ethylene selectivity with increase in temperature is due to the reason that higher reaction temperature favors water formation and this produced water distorts Cu and ZnO synergestics. (Ref. 2) A maximum of 179 ppm ethylene production is observed when the microwave reactor temperature reaches 200° C. with 10.1% CO₂ conversion and 0.9% H₂ conversion at 100-minute TOS. The production of ethylene is 1.6 times higher than other reaction temperatures. To check the reproducibility of the catalyst, the reaction for direct CO₂ hydrogenation to olefin is carried out in triplets over the modular catalyst system consisting of Cu/ZnO/Al₂O₃ and SAPO-34 catalyst. CO₂ conversion and ethylene production are expressed on a carbon basis as shown in FIG. 6B. A constant CO₂ conversion of 13.6±0.6% is observed after 50-minute TOS. Initially for the first 60 minutes, the reaction showed a higher level of variation in CO₂ conversion with 3% standard deviation error. With the increase in TOS, this error minimizes, and the conversion remained steady at 13%. Ethylene production is observed maximum with 175±37 ppm at 150-minute TOS. To access the change in the physical property of the Cu/Zn/Al₂O₃ after reaction under the microwave irradiation XRD analysis is performed. FIG. 7 presents the patterns of XRD of calcined and spent Cu/ZnO/Al₂O₃, (Refs. 27 and 31) showcasing discernible peculiarities.

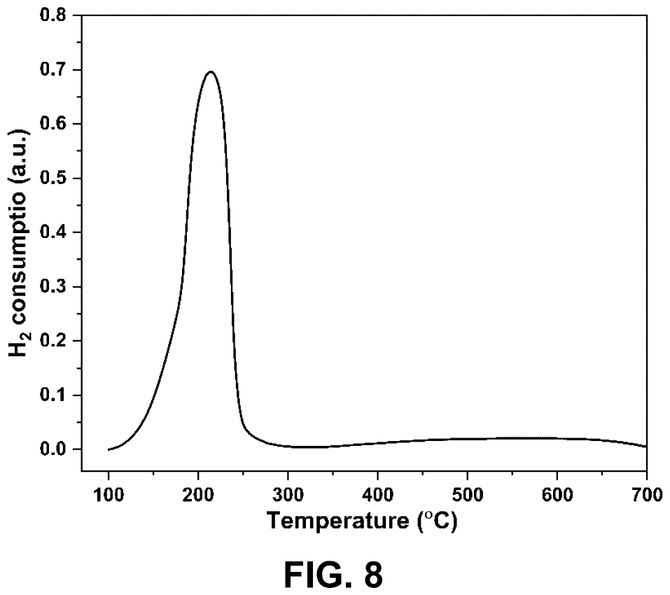

Experimental Methods: The microwave assisted CO₂ hydrogenation to methanol was conducted in a Sairem microwave system, equipped with a 2.45 GHz solid state generator and 0.94 kw power. Commercial catalyst Cu/ZnO/Al₂O₃ (HyKat SRK-50, CHEMPACK) was used in the reaction. A Micro-Epsilon pyrometer was positioned to measure the average temperature of the catalyst bed and control the microwave irradiation forward power. MeOH dehydration catalyst (SAPO-34, ACS Materials LLC, USA) was placed in the downstream of microwave system was heated using a furnace (Mellen, USA) equipped with a temperature control system. The feed gas mixture consisted of 60 vol. % H₂, 20 vol. % CO₂ balanced with N₂ for a total flow rate of 12 sccm is controlled using Brook mass flow controller. Prior to the hydrogenation reaction, Cu/ZnO/Al₂O₃ catalyst was reduced in gas mixture of 50 vol. % H₂ and 50 vol. % N₂ at 240° C. for 2 hours. The reduction temperature of the catalyst is measured by performing the temperature programmed reduction experiment. A profile of the temperature programmed reduction experiment is shown in FIG. 8. A back pressure regulator was used to maintain the pressure in the reactor system. To hold the high pressure, a thick wall quartz tube reactor (8 mm-ID, 12 mm-ID) was used to carry out the reaction. The composition of the reactor outlet was analyzed by online gas chromatography (4-channel Inficon Fusion micro-gas chromatograph). The reactor scheme of modular catalysis system is shown in FIG. 1. It consists of two different reactor systems. The microwave reactor system was employed to carry out the CO₂ hydrogenation reaction for methanol production over the commercial Cu/ZnO/Al₂O₃ catalyst (1.5 gram). In the downstream of microwave reactor, the methanol conversion to olefins was carried out over the SAPO-34 catalyst (0.3 gram).

Reduction behavior of the calcined Cu/ZnO/Al₂O₃ is investigated by H-temperature programmed reduction experiment (see FIG. 8). The sharp peak at 220° C. is attributed to the reduction of CuO. (Ref. 32) The broader peak between 400-700° C. was ascribed to the ZnO reduction by the hydrogen spillover due to adjacent Cu. (Ref. 33)

The data for conventional heating is recorded after 180 minutes of induction period. For conventional heating the CO₂ conversion is constant at ˜ 20% after 40-minute TOS (see FIG. 5A). Whereas the H₂ conversion decreases from ˜35% at 60-minute TOS to 20% at 140-minute TOS. The production of methanol with conventional heating increases with TOS. At 60-minute TOS, the MeOH production is 2206 ppm, and it increases to 4200 ppm at 140-minute TOS. Under microwave heating the induction period is about 60 minutes. The CO₂ conversion varies between 13% and 14%. H₂ conversion is constant at ˜17% after 40-minute TOS. A maximum of 1015 ppm methanol production is observed at 90-minute TOS under microwave heating. Based on these studies for methanol synthesis, it is evident that the benefit of using microwave heating is that it reduces the induction period to ⅓ of the required time and the CO₂ can be converted to useful chemicals at moderate temperature and pressure.

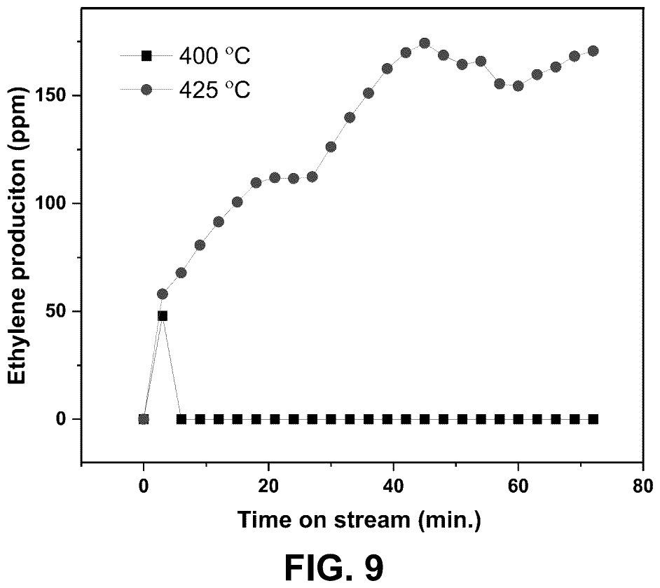

FIG. 9 shows the effect of MTO reaction temperature on ethylene production. When the temperature is 400° C., no ethylene is observed in the reactor outlet. Only methanol presence in outlet stream is verified using online gas chromatography. When the temperature is increased to 425° C. only ethylene is observed in the product stream. Based on this 425° C. reaction temperature is chosen for MTO reaction.

In the calcined catalyst sample, clearly represented in inset of FIG. 7 (log scale), it is interesting that the diffraction peaks of CuO (identified as JCPDS #00-041-0254) are prominently observed, whilst the diffraction peaks of ZnO (identified as JCPDS #01-079-0205) are less intense but still recognizable. The peaks corresponding to CuO are detected at 20 angles of 35.64°, 39.01°, 48.71°, 58.4° and 61.7°, whereas the peaks corresponding to ZnO are observed at 20 angles of 32.4°, 56.58°, 66.09° and 68.168°. These peaks correspond to the crystal planes of CuO with Miller indices of (−1 1 0), (2 0 0), (−2 0 2), (2 0 2), and (−1 1 3) respectively and ZnO with Miller indices of (1 0 0), (1 1 0), (2 0 0), and (1 1 2) respectively. All the peaks of CuO and ZnO are slightly shifted to the right due to the amorphous nature of alumina. The absence of any discernible peak in the alumina sample suggests that it exists in an amorphous state. The dispersing effect of alumina leads to a rise in the relative intensity of diffraction peaks as the value of alumina decreases. Furthermore, the evidence of a unique diffraction peak associated with graphite C (2θ=26.4°, JCPDS #0.01-089-8487) but very low intensity.

In other two catalyst samples at different temperatures, 160° C. and 200° C., the diffraction peaks associated with ZnO remain evident; however, the peaks related to CuO are no longer distinguishable. In contrast, the presence of distinct diffraction peaks at certain 2θ angles of 43.29°, 50.4°, 74.08°, 89.8°, and 95.04° can be observed, which are characteristic of metallic copper (PDF #03-065-9743). These peaks correspond to the crystal planes of copper with Miller indices of (1 1 1), (2 0 0), (2 2 0), (3 1 1), and (2 2 2) respectively. The aforementioned process denotes the comprehensive conversion of CuO to Cu by means of exposure to a gaseous environment consisting of hydrogen (H₂). Significantly, the condition of zinc oxide (ZnO) remains unchanged. Without wishing to be bound by theory, it can be supposed that Cu serves as the principal active site during the catalytic process involved in the synthesis of olefins from carbon dioxide. The evidence of a unique diffraction peak associated with graphite C (2θ=26.4°, JCPDS #0.01-089-8487) diminishes, which implies that there is no graphitic carbon present in the catalyst. Significantly, the distinct diffraction peaks associated with Al₂O₃ are absent in both reduced samples, indicating that Al₂O₃ may exist either in an amorphous state or in a highly scattered form within the catalyst matrix. (Refs. 34 and 35)

Conclusion: In this example, the hydrogenation of CO₂ to methanol and olefins was studied in a modular catalytic over the Cu/ZnO/Al₂O₃ and SAPO-34 catalysts. It was found that highly thermodynamic stable CO₂ molecules can be hydrogenated to methanol under milder operating conditions (80 psig and 200° C.) when the catalyst surface is heated using microwave irradiation. A maximum methanol production of 1015 ppm is observed. Microwave heating offers several benefits, including the reduction of initial activation period of hydrogenation catalyst. It reduces the induction period of Cu/ZnO/Al₂O₃ by ⅓ of the required time when heated with conventional thermal furnace. The performance for CO₂ hydrogenation to olefins is carried out in the modular reactor. At 80 psig and 200° C. a maximum C2 olefin production (˜170 ppm) was achieved at 3:1 H₂/CO₂ ratio. The potential of microwave-driven catalytic technology to hydrogenate the CO₂ using green hydrogen to valuable chemical is feasible in a two-zone single reactor operated under the relatively mild operating conditions.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method, comprising:
flowing a gas mixture over a first catalyst;
heating the first catalyst by microwave irradiation to a first target temperature, thereby producing a first gaseous product comprising methanol;
flowing the first gaseous product over a molecular sieve catalyst;
heating the molecular sieve catalyst to a second target temperature, thereby producing a second product comprising at least one hydrocarbon compound;
wherein the gas mixture comprises CO₂ and H₂;
wherein the first catalyst comprises a first metallic nanoparticle and a second metallic nanoparticle on a support;
wherein the first metallic nanoparticle is selected from Cu, Cr, Co, Pd, and a combination thereof;
wherein the second metallic nanoparticle is selected from ZnO, Cr₂O₃, In₂O₃, and a combination thereof; and
wherein the support comprises Al₂O₃, MgO, SiO₂—Al₂O₃, or Cr₂O₃.

2. The method of claim 1, wherein the method is carried out a pressure of about 1 bar to about 100 bar.

3. The method of claim 1, wherein the molecular sieve catalyst is a zeolitic molecular sieve catalyst.

4. The method of claim 3, wherein the zeolitic molecular sieve catalyst comprises SAPO-18, SAPO-34, SAPO-35, SAPO-44, SAPO-56, ZSM-5, or a combination thereof.

5. The method of claim 1, wherein the first target temperature is greater than about 160° C.

6. The method of claim 1, wherein the first target temperature is from about 160° C. to about 500° C.

7. The method of claim 1, wherein the first target temperature is from about 160° C. to about 400° C.

8. The method of claim 1, wherein the second target temperature is from about 380° C. to about 500° C.

9. The method of claim 1, wherein the second target temperature is from about 400° C. to about 500° C.

10. The method of claim 1, wherein the gas mixture comprises CO₂ and H₂ in a volume ratio of about 1:10 to about 1:1.

11. The method of claim 1, wherein the at least one hydrocarbon compound comprises an olefin.

12. The method of claim 1, wherein the at least one hydrocarbon compound comprises a light olefin.

13. The method of claim 1, wherein the at least one hydrocarbon compound comprises a C5-C25 hydrocarbon.

14. The method of claim 1, wherein the at least one hydrocarbon compound comprises a C1-C4 alcohol.

15. The method of claim 1, wherein the at least one hydrocarbon compound comprises a higher alcohol.

16. The method of claim 1, wherein the at least one hydrocarbon compound comprises a hydrocarbon fuel.

17. The method of claim 1, wherein the at least one hydrocarbon compound comprises a hydrocarbon fuel selected from gasoline, jet fuel, diesel, and a combination thereof.

18. A composition, comprising an olefin produced using the method of claim 1.

19. A composition, comprising a hydrocarbon fuel produced using the method of claim 1.

20. The composition of claim 19, wherein the hydrocarbon fuel is selected from gasoline, jet fuel, diesel, and a combination thereof.
